(19) United States
(12) Patent Application Publication
(10) Pub. No.: US 2008/0003624 A1
(43) Pub. Date: Jan. 3, 2008

(54) Cancer metastasis determination method

(75) Inventors: Hideki Takata, Kobe (JP); Kayo Hiyama, Kobe (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 11/808,307

(22) Filed: Jun. 8, 2007

(30) Foreign Application Priority Data
Jun. 13, 2006 (JP) 2006-163578

(51) Int. Cl. G01N 33/574; C12Q 1/04; G01N 33/50

(52) U.S. Cl. 435/7.23; 435/34; 435/40.52

(57) ABSTRACT

A method of determining metastasis of a cancer to a lymph node, comprising the steps of: quantitating a polypeptide associated with cytokeratin, in a sample for detection prepared using a lymph node tissue collected from a living body, and determining metastasis of a cancer cell to the lymph node based on the quantitation result of the polypeptide is provided. Thereby, metastasis of a cancer to a lymph node can be determined with high reliance.

### FIELD OF THE INVENTION

The present invention relates to a method of determining metastasis of a cancer cell to a lymph node.

### BACKGROUND

Previously, in order to determine metastasis of a cancer cell to a lymph node, a lymph node tissue has been collected from a living body, and an expression amount (protein or mRNA) of a tumor marker gene in this lymph node tissue has been measured. When an expression amount of a tumor marker gene is more (or less) than an expression amount of a normal cell, it is suspected that a cancer cell has been metastasized to the lymph node tissue.

As a method of determining metastasis of a cancer to a lymph node, for example, the method described in JP2006053113 is known. JP2006053113 describes a method of diagnosing metastasis of pulmonary adenocarcinoma to a lymph node. Specifically, a tissue piece of lung of a patient with lymph node metastasis, and a tissue piece of lung of a patient without lymph node metastasis are used as a sample, and an expression amount of various proteins is measured, thereby, a protein useful as a metastasis diagnosis marker is identified. In addition, there is disclosure that, by using this protein, metastasis of a lung cancer in a pulmonary adenocarcinoma patient to a lymph node can be diagnosed.

### SUMMARY

The scope of the present invention is defined solely by the appended claims, and is not affected to any degree by the statements within this summary.

The present inventors found that, upon determination of lymph node metastasis, by measuring a protein of a tumor marker contained not in a primary focus but in a lymph node tissue, lymph node metastasis can be determined with high reliance, which resulted in completion of the invention.

An object of the present invention is to provide a method which can determine metastasis of a cancer to a lymph node with high reliance, and a reagent kit used therefor.

The present invention provides a method of determining metastasis of a cancer to a lymph node, comprising a step of quantitating a polypeptide associated with cytokeratin in a sample for detection prepared using a lymph node tissue collected from a living body, and a step of determining metastasis of a cancer cell to the lymph node based on quantitation result of the polypeptide.

According to the present invention, a method of determining metastasis of a cancer to a lymph node is provided. In accordance with this method, metastasis of a cancer to a lymph node can be determined with higher reliance.

### BRIEF DESCRIPTION OF THE DRAWINGS

FIG. 1 is a graph showing the result of Example 1.

FIG. 2 is a graph showing the result of Example 2.

FIG. 3 is a graph showing the result of Example 3.

FIG. 4 is a graph showing the result of Example 4.

### DETAILED DESCRIPTION OF THE EMBODIMENT

A method of determining metastasis of a cancer to a lymph node which is one embodiment of the present invention comprises a quantitation step of quantitating a polypeptide associated with cytokeratin in a sample for detection prepared using a lymph node tissue collected from a living body, and a determination step of determining metastasis of a cancer cell to the lymph node based on quantitation result of the polypeptide.

As used herein, a cancer is a tumor which has became malignant, and has the same meaning as that of a malignant tumor. The cancer includes carcinoma, sarcoma, and hematopoietic organ-derived cancer. Examples of the carcinoma include epithelial cell-derived cancers such as a breast cancer, a stomach cancer, a colon cancer, a prostate cancer, a cervical cancer, and a cancer of uterine body. Examples of the sarcoma include osteosarcoma and soft-tissue sarcoma. Examples of the hematopoietic organ-derived cancer include leukemia and malignant lymphoma.

In addition, as used herein, “metastasis of cancer” is metastasis of a cancer cell present in a primary focus to a tissue different from the primary focus, and is used as having the same meaning as that of “metastasis of cancer cell”.

As a sample, a lymph node tissue is used. As used herein, a lymph node tissue includes all or a part of a lymph node tissue excised by operation or the like, and a sample containing a plurality of lymph node cells collected from a lymph node by biopsy or the like.

According to the method of the present embodiment, whether a cancer cell is contained in a lymph node tissue or not can be determined. That is, whether cancer cells has been metastasized to a lymph node tissue from a primary focus or not can be determined. This determination result can be used as one index upon determination of therapy policy or tissue dissection region. For example, the determination result in accordance with the method of the present embodiment in a lymph node tissue near a tumor of a breast cancer, a stomach cancer or a colon cancer patient collected by biopsy assists determination of necessity of isolation of a lymph node, a degree of dissection, and the like.

Upon quantitation of a polypeptide, a sample for detection is prepared from a lymph node tissue. The polypeptide contained in this sample for detection is then quantitated. This polypeptide is associated with cytokeratin. For example, the polypeptide may be a protein of cytokeratin (hereinafter, also referred to as CK), or a fragment thereof. CK is also called keratin, and one of proteins constituting a cytoskeleton. In CK, there are kinds such as CK18, 19 and 20, and any kind of CK may be quantitated, CK19 being preferable.

A method of preparing a sample for detection is not particularly limited, but a commercially available protein extraction kit can be used. It is preferable to perform preparation using a buffer. Specifically, a buffer is added to a lymph node tissue. Then, cells in a buffer are chemically and/or physically treated. Thereby, a polypeptide in cells is transferred into a solution. This solution containing the polypeptide can be used as a sample for detection.

It is preferable that this buffer is strongly acidic. A preferable range of a pH is 2.5 to 5.0, and a more preferable range is 3.0 to 4.0. In order to retain a pH in this range, the known buffers can be used.

In addition, it is preferable that the buffer contains a surfactant. The reason is as followed: a cell membrane or a nuclear membrane is damaged with the surfactant. Therefore, a molecule in a cell is more easily transferred into a solution via damage. A kind of the surfactant is not particularly limited as far as it has such the action. A preferable surfactant is a nonionic surfactant. A polyoxyethylene nonionic surfactant is more preferable. Particularly, a polyoxyethylene nonionic surfactant represented by the following general formula:
R₁-R₂-(CH₂CH₂O)ₙ—H
(wherein R₁ is an alkyl group, an alkenyl group or an alkynyl group having 10 to 22 carbon atoms, or an isooctyl group; R₂ is —O— or —(C₆H₆)—O—; n is an integer of 8 to 120) is preferable.

Examples of the polyoxyethylene nonionic surfactant include polyoxyethylene laryl ether, polyoxyethylene cetyl ether, polyoxyethylene oleyl ether, polyoxyethylene myristyl ether, polyoxyethylene stearyl ether, polyoxyethylene nonyl phenyl ether, and polyoxyethylene isooctyl phenyl ether.

More specifically, Brij35 (polyoxyethylene (35) lauryl ether) is preferable. A concentration of the surfactant in the buffer is preferably 0.1 to 6% (v/v), more preferably 1 to 5% (v/v).

The buffer may further contain dimethyl sulfoxide (DMSO). A concentration of DMSO in the buffer is preferably 1 to 50% (v/v), more preferably 5 to 30% (v/v), most preferably 10 to 25% (v/v).

By using a buffer containing dimethyl sulfoxide and the surfactant and having the aforementioned pH in preparing the sample for detection, a protein present in a cell such as cytokeratin can be effectively transferred into a solution. It is presumed that this effect is generated by a composite factor such as a composition and a pH of the buffer.

Upon preparation of the sample for detection, it is preferable that a lymph node tissue is subjected to physical treatment such as homogenization and the like. Thereby, a cell membrane and a nuclear membrane of a cell in a biological sample are physically ground, and a molecule in the cell is easily transferred into a solution. Homogenization may be performed manually with a pestle, or may be performed using a commercially available electric-powered homogenizer. By centrifuging the resulting homogenate for a few seconds to a few minutes, a broken piece of a cell suspended in the homogenate can be precipitated. Thereby, a supernatant containing a molecule present in a cell such as a protein and the like can be used as the sample for detection.

When the aforementioned buffer is used, adjustment can be performed only by simple centrifugation operation. For this reason, treatment such as purification of a protein and the like which have previously been necessary becomes unnecessary. Therefore, preparation of the sample for detection can be performed simply and in a short time.

Further, the sample for detection also contains a DNA and an RNA. Therefore, by using the same sample for detection, quantitation of a specified nucleic acid (e.g. mRNA of tumor marker) and detection of mutation of a specified gene (e.g. one gene polymorphism) can be also performed.

Quantitation of the polypeptide can be performed by the known method, and is not particularly limited, but it is preferable to use a quantitation method utilizing an antigen antibody reaction (immunoassay method). For example, an analysis method using a protein chip or the like, an immunoblotting method such as a dot blotting method and a Western blotting method, radioimmunoassay (RIA), enzyme immunoassay (EIA), fluorescent immunoassay (FIA), chemiluminescent immunoassay (CLIA), and counting immunoassay (CIA: See Sysmex Journal Vol. 20 No. 1,77-86(1997)) canbe used. Among them, when a Western blotting method is used, kinds of polypeptides contained in the sample for detection can be separated based on a molecular weight. Therefore, an objective polypeptide can be quantitated more specifically.

It is preferable that a quantitation value of the polypeptide is calculated based on a pre-produced calibration curve. The calibration curve can be produced by measurement under the same condition as that of polypeptide measurement of the sample for detection using a sample containing the known amount of a protein.

Based on the quantitation result of the above-measured polypeptide, the presence or the absence of metastasis of a cancer to a lymph node tissue is determined. It is preferable that this determination is performed by comparing a quantitation value of the polypeptide measured by the aforementioned method with a predetermined threshold.

The threshold is a value which is appropriately set depending on kinds of a cancer and cytokeratin. For example, the threshold can be set at a value which is not higher than an amount of a polypeptide contained in a lymph node tissue for which metastasis of a cancer cell has been recognized (positive sample) and is higher than an amount of a polypeptide contained in a lymph node tissue for which metastasis of a cancer cell has not been recognized (negative sample). Particularly, it is preferable that an amount of a polypeptide of a plurality of positive samples and an amount of a polypeptide of a plurality of negative samples are pre-measured, and a value by which these positive sample and negative sample can be discriminated at a highest probability is set as a threshold.

The method of the present embodiment can detect cancer metastasis at the equivalent precision to that of determination by the previously performed tissue diagnosis and nucleic acid detection. Further, this method is a more objective method having a lower possibility of overlooking cancer metastasis than that of tissue diagnosis which makes determination by one cross-section of a tissue.

A reagent kit used in determination of a metastasis of a cancer to a lymph node which is one embodiment of the present invention has the aforementioned buffer, and an antibody used in an immunoassay method. This reagent kit can be suitably used in the aforementioned method of determination of lymph node metastasis.

### EXAMPLE

### Example 1

1. Preparation of Measurement Sample

Using 113 lymph nodes excised from breast cancer patients, measurement samples were prepared.

Among these lymph nodes, 91 were cancer metastasis negative lymph nodes (among them, 37 are pN0, and 54 were pN1-3), and 22 were cancer metastasis positive lymph nodes (pN+) in microscopy of a tissue section (tissue diagnosis) stained with hematoxylin-eosin.

First, to each lymph node (about 50 to 600 mg/node) was added 200 μL of a buffer of a pH3.4 (containing 200 mM glycin-HCl,5% Brij35 (polyoxyethylene (35) lauryl ether, manufactured by SIGMA) and 20% DMSO (Wako Pure Chemical Industries, Ltd.)), and this was homogenized with a blender. The resulting homogenate was centrifuged at 10,000× g at room temperature for 1 minute, and 50 to 200 μL of a supernatant was collected, which was used as a sample for detection.

2. Quantitation of CK19 Protein

To 20 μL of the sample for detection was added 10 μL of 3×SDS-treated buffer (containing 150 mM Tris HCl (pH6.8), 300 mM dithiothreitol, 6% sodium dodecylsulfate (SDS), 0.3% bromophenol blue, and 30% glycerol), and this was warmed at 95° C. for 5 minutes to prepare an electrophoresis sample. Using 15 μL of this electrophoresis sample, SDS-PAGE was performed. A gel after electrophoresis was transferred to a PVDF membrane, this was immersed in a blocking buffer (containing 20 mM Tris (pH7.6), 137 mM NaCl, 0.1% Tween20, and 5% skimmilk), and shaken at room temperature for 1 hour, thereby, blocking treatment of the membrane was performed. After blocking treatment, the membrane was washed with TBS-T (containing 20 mMTris (pH7.6), 137 mM NaCl, and 0.1% Tween20) for 2 minutes. Then, the membrane was immersed in a primary antibody solution (a solution obtained by 1/500 diluting primary antibody: ant-cytokeratin 19 antibody (A53-B/A2) (manufactured by Santa Cruz, Lot.#L2403: recognizing position 312 to 335 of CK19 protein amino acid sequence) with TBS-T), and allowed to stand at 4° C. overnight to perform an antibody reaction. After the reaction, this membrane was washed with TBS-T four times four 5 minutes, and the membrane was immersed in a solution of a secondary antibody which can bind to the primary antibody and is conjugated with horseradish peroxidase as a labeling enzyme (a solution obtained by 1/2000 diluting a secondary antibody: ECL anti-mouse IgG HRP linked F (ab′)2 fragment (manufactured by GE Health Care Bioscience) with TBS-T), and allowed to stand at room temperature for 30 minutes to perform an antibody reaction. After the reaction, the membrane was washed with TBS-T four times for 5 minutes, and an enzyme reaction was performed with ECL-Advanced Western Blotting Detection Kit (manufactured by GE Health Care Bioscience). An image of the membrane after the enzyme reaction was taken with LumiAnalyst (manufactured by Roche), and a fluorescent intensity of the membrane image was calculated with QuantityOne (manufactured by Biorad). The above-measured fluorescent intensity was fitted into a calibration curve to calculate an amount of the CK19 protein. The calibration curve was produced by accommodating a sample containing a CK19 protein having the known concentration in a well different from a well accommodating a measurement sample with the same gel, and performing the fluorescent intensity measurement.

3. Result

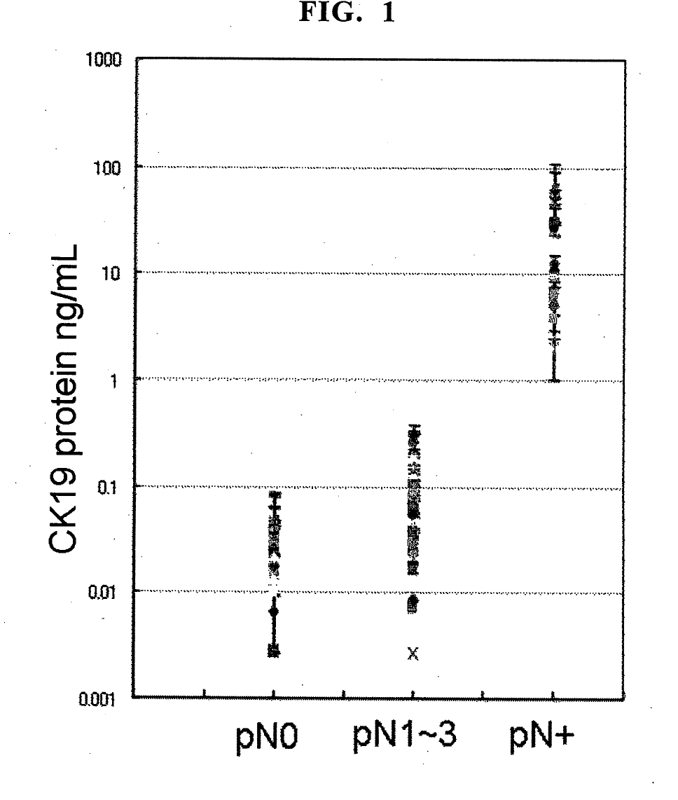

The measured protein quantitated value (concentration: ng/μL) was developed on a logarithmic axis to produce a graph. This graph is shown in FIG. 1. In the graph, “pN0” indicates a quantitated value of the CK19 protein in the sample for detection prepared using a lymph node of pN0. In addition, “pN1-3” indicates a quantitated value of the CK19 protein in the sample for detection prepared using lymph nodes of pN1-3. Further, “pN+” indicates a quantitated value of the CK19 protein in the sample for detection prepared using a cancer metastasis positive lymph node.

As seen from FIG. 1, a quantitated value of the CK19 protein measured in the present Example was consistent with determination by histological examination.

In addition, among cancer metastasis negative lymph nodes, in a lymph node in which a mass of the CK19 protein was highest, 0.3 ng/μL of the CK19 protein was detected. Further, among cancer metastasis positive lymph nodes, in a lymph node in which a mass of the CK19 protein was lowest, 1 ng/μL of the CK19 protein was detected. That is, in order to determine the presence or the absence of cancer metastasis, a threshold can be set between 0.3 and 1. Therefore, when a quantitated value of the CK19 protein was not lower than this threshold, it can be determined that metastasis of a cancer to a lymph node is positive. Conversely, when the quantitated value is lower than the threshold, it can be determined that metastasis of a cancer to a lymph node is negative.

From the forgoing, it was demonstrated that, according to the method of the present example, metastasis of a cancer to a lymph node could be detected at an equivalent precision to that of tissue diagnosis which has previously been performed widely.

### Example 2

1. Preparation of Measurement Sample

Using 31 lymph nodes excised from breast cancer patients, samples for detection were prepared as in Example 1. Among them, in microscopy of a tissue section stained with hematoxylin-eosin (tissue diagnosis), lymph nodes for which metastasis of a cancer cell was recognized (positive sample) was 22, and lymph nodes for which metastasis of a cancer cell was not recognized (negative sample) was 9.

2. Quantitation of CK19 Protein

Using the above samples for detection, the CK19 protein was quantitated as in Example 1.

3. Quantitation of CK19 mRNA

Using the above samples for detection, an mRNA of CK19 in samples was quantitated. First, the sample for detection was set in a nucleic acid amplification apparatus (GD-100, manufactured by Sysmex), and a cDNA of CK19 was amplified by a RT-LAMP reaction (as a measurement reagent, in GD-100, a cytokeratin reagent (manufactured by Sysmex) was set as a nucleic acid amplification reagent). By measuring a turbidity of the reaction solution at real time, an mRNA of CK19 (copy number per 1 μL of sample for detection) was quantitated.

4. Result

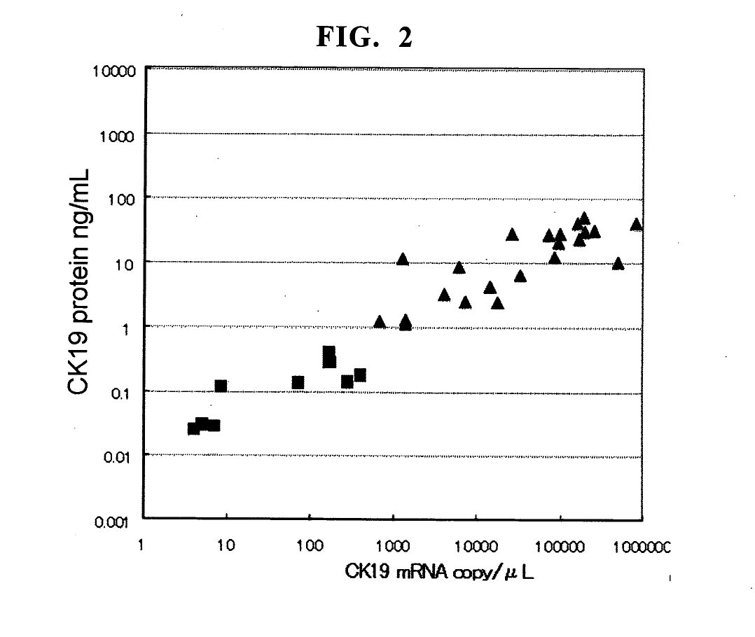

The measurement mRNA quantitated value (concentration: copy number/μL) and the protein quantitated value (concentration: ng/μL) were developed on a logarithmic axis to produce a graph. This graph is shown in FIG. 2. In the graph, ▴ is a positive sample which was histologically determined to be cancer metastasis positive, and ▪ is a negative sample which was histologically determined to be cancer metastasis negative.

From FIG. 2, the quantitation result of the CK19 protein measured in the present example was well correlated with the quantitation result of an mRNA of CK19. In addition, by setting a threshold for a quantitated value of the CK19 protein between 0.3 and 1, all positive samples could be determined to be cancer metastasis positive. Therefore, it was demonstrated that, according to the method of the present example, metastasis of a cancer to a lymph node could be detected at an equivalent precision to that of determination by detection of an mRNA which is said to be highly sensitive.

From the forgoing, according to the method of the present embodiment, metastasis of a cancer to a lymph node can be detected at an equivalent precision as that of the conventional method. In addition, the result consistent with the determination result of cancer metastasis by tissue diagnosis and the determination result of cancer metastasis by nucleic acid detection can be obtained.

The foregoing detailed description and examples have been provided by way of explanation and illustration, and are not intended to limit the scope of the appended claims. Many variations in the presently preferred embodiments will be obvious to one of ordinary skill in the art, and remain within the scope of the appended claims and their equivalents.

### Example 3

1. Preparation of Measurement Sample

Using 123 lymph nodes excised from colon cancer patients, measurement samples were prepared.

Among these lymph nodes, 92 were cancer metastasis negative lymph nodes (among them, 31 are pN0, and 61 were pN1-3), and 31 were cancer metastasis positive lymph nodes (pN+) in microscopy of a tissue section (tissue diagnosis) stained with hematoxylin-eosin. Measurement sample were prepared by using same method described in Example 1.

2. Quantitation of CK19 Protein

Using the above samples for detection, the CK19 protein was quantitated as in Example 1.

3. Result

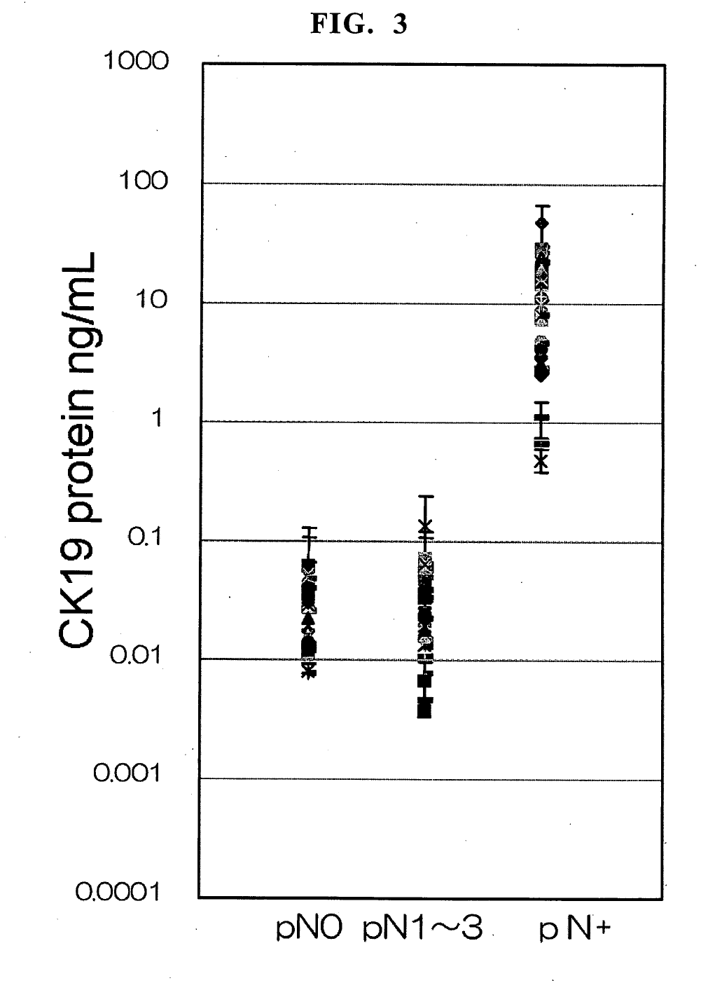

As seen from FIG. 3, a quantitated value of the CK19 protein measured in the present Example was also consistent with determination by histological examination in cases using a colon cancer.

### Example 4

1. Preparation of Measurement Sample

Using 98 lymph nodes excised from stomach cancer patients, measurement samples were prepared.

Among these lymph nodes, 72 were cancer metastasis negative lymph nodes (among them, 34 are pN0, and 38 were pN1-2), and 26 were cancer metastasis positive lymph nodes (pN+) in microscopy of a tissue section (tissue diagnosis) stained with hematoxylin-eosin. Measurement sample were prepared by using same method described in Example 1.

2. Quantitation of CK19 Protein

Using the above samples for detection, the CK19 Protein was quantitated as in Example 1.

3. Result

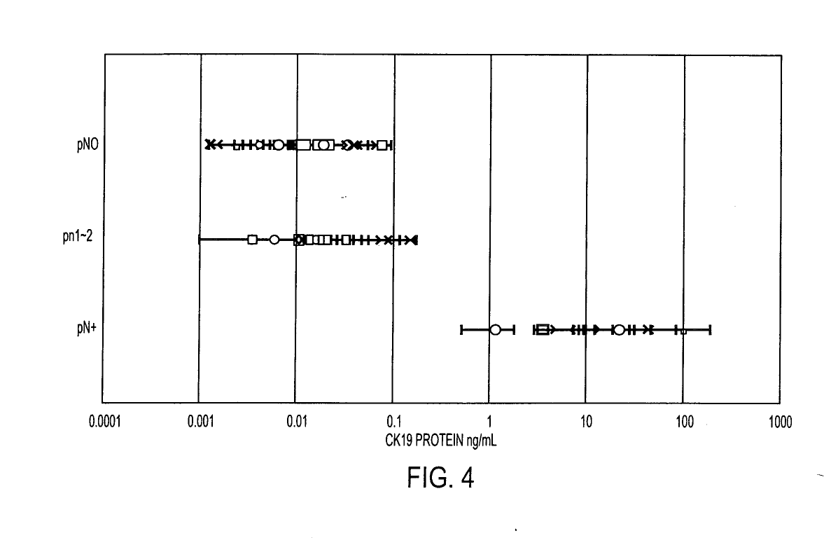

As seen from FIG. 4, a quantitated value of the CK19 protein measured in the present Example was also consistent with determination by histological examination in cases using a stomach cancer.

What is claimed is:

1. A method of determining metastasis of a cancer to a lymph node, comprising the steps of:
quantitating a polypeptide of cytokeratin, in a sample for detection prepared using a lymph node tissue collected from a living body, and
determining whether metastasis of a cancer cell to the lymph node or not based on the quantitation result of the polypeptide.

2. The method according to claim 1, wherein the sample for detection is a solution obtained by mixing the lymph node tissue with a buffer, and grinding the lymph node tissue in the buffer.

3. The method according to claim 2, wherein the buffer has a pH of 2.5 to 5.0.

4. The method according to claim 2, wherein the buffer contains dimethyl sulfoxide.

5. The method according to claim 2, wherein the buffer contains a nonionic surfactant.

6. The method according to claim 2, wherein the buffer contains a polyoxyethylen nonionic surfactant.

7. The method according to claim 1, wherein the quantitation step is performed so as to quantitate the polypeptide by immunoassay using anti-cytokeratin antibody.

8. The method according to claim 1, wherein the determination step is performed so as to determine the metastasis of a cancer cell by comparing the quantitation result with a predetermined threshold.

9. The method according to claim 8, wherein the determination step is performed so as to determine that a cancer cell has been metastasized to the lymph node tissue when the quantitation result is not lower than the threshold.

10. The method according to claim 1, wherein the cytokeratin is cytokeratin 19.

11. The method according to claim 1, wherein the cancer cell is a carcinoma cell.

12. The method according to claim 1, wherein the cancer cell is a breast cancer cell, a stomach cancer cell or a colon cancer cell.

13. A method of determining metastasis of a cancer to a lymph node, comprising the steps of:
mixing a lymph node tissue excised from a cancer patient with a buffer,
grinding the lymph node tissue in the buffer to prepare a sample for detection,
quantitating a polypeptide of cytokeratin, in the sample for detection,
comparing the quantitation result with a predetermined threshold, and
determining whether metastasis of a cancer cell to the lymph node or not based on the comparison result.

14. A reagent kit used in determination of metastasis of a cancer to a lymph node, comprising:
a buffer for mixing with a lymph node tissue collected from a living body to prepare a sample for detection, and
an antibody for quantitating a polypeptide of cytokeratin using an immunoassay method.

15. The reagent kit according to claim 14, wherein the buffer has a pH of 2.5 to 5.0.

16. The reagent kit according to claim 14, wherein the buffer contains dimethyl sulfoxide.

17. The reagent kit according to claim 14, wherein the buffer contains a nonionic surfactant.

18. The reagent kit according to claim 14, wherein the buffer contains a polyoxyethylene nonionic surfactant.

19. The reagent kit according to claim 14, wherein the cytokeratin is cytokeratin 19.

20. The reagent kit according to claim 14, wherein the immunoassay method is an immunoblotting method.
